(19) United States
(12) Patent Application Publication
(10) Pub. No.: US 2006/0247296 A1
(43) Pub. Date: Nov. 2, 2006

(54) Methods of using and compositions comprising (+)-3-(3,4-dimethoxy-phenyl)-3-(1-oxo-1,3-dihydro-isoindol-2-yl)-propionamide

(76) Inventors: George W. Muller, Bridgewater, NJ (US); Roger Shen-Chu Chen, Edison, NJ (US)

Correspondence Address: Jones Day, 222 East 41st Street, New York, NY, 10017, US

(21) Appl. No.: 10/535,004

(22) PCT Filed:
(86) PCT No.: PCT/US03/36740

Related U.S. Application Data
(60) Provisional application No. 60/427,379, filed on Nov. 18, 2002.

(51) Int. Cl. A61K 31/4035

(52) U.S. Cl. 514/417

(57) ABSTRACT

Enantiomerically pure (+)-3-(3,4-dimethoxy-phenyl)-3-(1-oxo-1,3-dihydroisoindol-2-yl)-propionamide, and pro-drugs, metabolites, polymorphs, salts, solvates (e.g. hydrates), and clathrates thereof are discussed. Methods of treating and/or preventing various diseases and disorders, such as those ameliorated by the reduction of levels of TNF-α or the inhibition of PDE4, are also disclosed.

This application claims the benefit of U.S. provisional application No. 60/427,379, filed Nov. 18, 2002, the entirety of which is incorporated herein by reference.

### 1. FIELD OF INVENTION

The invention relates to methods of using and pharmaceutical compositions comprising enantiomerically pure (+)-3-(3,4-dimethoxy-phenyl)-3-(1-oxo-1,3-dihydro-isoindol-2-yl)-propionamide. More particularly, the present invention is directed to the inhibition of tumor necrosis factor alpha (TNF-α) production and/or phosphodiesterase type 4 (PDE4) activity by administration of (+)-3-(3,4-dimethoxy-phenyl)-3-(1-oxo-1,3-dihydro-isoindol-2-yl)-propionamide. The compound, which may be used in the methods and compositions of the invention, is capable of treating or preventing cancer, inflammatory and autoimmune diseases and disorders. In one embodiment, the invention is directed to the combined use of (+)-3-(3,4-dimethoxy-phenyl)-3-(1-oxo-1,3-dihydro-isoindol-2-yl)-propionamide and a second active agent for the prevention or treatment of cancer, inflammatory or autoimmune diseases or disorders.

### 2. BACKGROUND OF THE INVENTION

Tumor necrosis factor alpha (TNF-α) is a cytokine that is released primarily by mononuclear phagocytes in response to immunostimulators. TNF-α is capable of enhancing most cellular processes, such as differentiation, recruitment, proliferation, and proteolytic degradation. At low levels, TNF-α confers protection against infective agents, tumors, and tissue damage. However, TNF-α also has role in many diseases. When administered to mammals such as humans, TNF-α causes or aggravates inflammation, fever, cardiovascular effects, hemorrhage, coagulation, and acute phase responses similar to those seen during acute infections and shock states. Enhanced or unregulated TNF-α production has been implicated in a number of diseases and medical conditions, for example, cancers, such as solid tumors and blood-born tumors; heart disease, such as congestive heart failure; and viral, genetic, inflammatory, allergic, and autoimmune diseases.

T-cells are a class of white blood cells that play an important role in the immune response, and help protect the body from viral and bacterial infections. Diminished T-cell levels strongly contribute to the inability of HIV patients to combat infections, and abnormally low T-cell levels are prominent in a number of other immune deficiency syndromes, including DiGeorge Syndrome, and in certain forms of cancer, such as T-cell lymphoma.

Cancer is a particularly devastating disease, and increases in blood TNF-α levels are implicated in the risk of and the spreading of cancer. Normally, in healthy subjects, cancer cells fail to survive in the circulatory system, one of the reasons being that the lining of blood vessels acts as a barrier to tumor-cell extravasation. However, increased levels of cytokines have been shown to substantially increase the adhesion of cancer cells to endothelium in vitro. One explanation is that cytokines, such as TNF-α, stimulate the biosynthesis and expression of a cell surface receptors called ELAM-1 (endothelial leukocyte adhesion molecule). ELAM-1 is a member of a family of calcium-dependent cell adhesion receptors, known as LEC-CAMs, which includes LECAM-1 and GMP-140. During an inflammatory response, ELAM-1 on endothelial cells functions as a “homing receptor” for leukocytes. ELAM-1 on endothelial cells was shown to mediate the increased adhesion of colon cancer cells to endothelium treated with cytokines (Rice et al., 1989, Science 246:1303-1306).

Inflammatory diseases such as arthritis, related arthritic conditions (e.g., osteoarthritis and rheumatoid arthritis), inflammatory bowel disease, sepsis, psoriasis, chronic obstructive pulmonary diseases and chronic inflammatory pulmonary diseases are also prevalent and problematic ailments. TNF-α and PDE4 play a central role in the inflammatory response and the administration of their antagonists block chronic and acute responses in animal models of inflammatory disease.

Enhanced or unregulated TNF-α production has been implicated in viral, genetic, inflammatory, allergic, and autoimmune diseases. Examples of such diseases include, but are not limited to: HIV; hepatitis; adult respiratory distress syndrome; bone-resorption diseases; chronic obstructive pulmonary diseases; chronic pulmonary inflammatory diseases; dermatitis; cystic fibrosis; septic shock; sepsis; endotoxic shock; hemodynamic shock; sepsis syndrome; post ischemic reperfusion injury; meningitis; psoriasis; fibrotic disease; cachexia; graft rejection; auto-immune disease; rheumatoid spondylitis; arthritic conditions, such as rheumatoid arthritis and osteoarthritis; osteoporosis; inflammatory-bowel disease; Crohn's disease; ulcerative colitis; multiple sclerosis; systemic lupus erythrematosus; leprosy (e.g., ENL); radiation damage; asthma; and hyperoxic alveolar injury. Tracey et al., 1987, Nature 330:662-664 and Hinshaw et al., 1990, Circ. Shock 30:279-292 (endotoxic shock); Dezube et al., 1990, Lancet, 335:662 (cachexia ); Millar et al., 1989, Lancet 2:712-714 and Ferrai-Baliviera et al., 1989, Arch. Surg. 124:1400-1405 (adult respiratory distress syndrome); Bertolini et al., 1986, Nature 319:516-518, Johnson et al., 1989, Endocrinology 124:1424-1427, Holler et al., 1990, Blood 75:1011-1016, and Grau et al., 1989, N. Engl. J. Med. 320:1586-1591 (bone resorption diseases); Pignet et al., 1990, Nature, 344:245-247, Bissonnette et al., 1989, Inflammation 13:329-339 and Baughman et al., 1990, J. Lab. Clin. Med. 115:36-42 (chronic pulmonary inflammatory diseases); Elliot et al., 1995, Int. J. Pharmac. 17:141-145 (rheumatoid arthritis); von Dullemen et al., 1995, Gastroenterology, 109:129-135 (Crohn's disease); Duh et al., 1989, Proc. Nat. Acad. Sci. 86:5974-5978, Poll et al., 1990, Proc. Nat. Acad. Sci. 87:782-785, Monto et al., 1990, Blood 79:2670, Clouse et al., 1989, J. Immunol. 142, 431-438, Poll et al., 1992, AIDS Res. Hum. Retrovirus, 191-197, Poli et al. 1990, Proc. Natl. Acad. Sci. 87:782-784, Folks et al., 1989, PNAS 86:2365-2368 (HIV and opportunistic infections resulting from HIV).

Adenosine 3′,5′-cyclic monophosphate (cAMP) also plays a role in many diseases and conditions, such as, but not limited to respiratory diseases, asthma and inflammation (Lowe and Cheng, Drugs of the Future, 17(9), 799-807, 1992). It has been shown that the elevation of cAMP in inflammatory leukocytes inhibits their activation and the subsequent release of inflammatory mediators, including TNF-α and nuclear factor κB (NF-κB). Increased levels of cAMP also lead to the relaxation of airway smooth muscle.

It is believed that a primary cellular mechanism for the inactivation of cAMP is the breakdown of cAMP by a family of isoenzymes referred to as cyclic nucleotide phosphodiesterases (PDE) (Beavo and Reitsnyder, Trends in Pharm., 11, 150-155, 1990). There are eleven known members of the family of PDEs. It is recognized that the inhibition of PDE type IV (PDE4) is particularly effective in both the inhibition of inflammatory mediated release and the relaxation of airway smooth muscle (Verghese, et al., Journal of Pharmacology and Experimental Therapeutics, 272(3), 1313-1320, 1995; and Torphy, Amer. J. Resp. Crit. Care Med., 157, 351-70, 1998). Thus, compounds that specifically inhibit PDE4 inhibit inflammation and aid the relaxation of airway smooth muscle with a minimum of unwanted side effects, such as cardiovascular or anti-platelet effects.

Accordingly, compounds that can block the activity or inhibit the production of certain cytokines including TNF-α may be useful in the treatment and prevention of various diseases. See, e.g., Lowe, 1998 Exp. Opin. Ther. Patents 8:1309-1332. One such compound is racemic 3-(3,4-dimethoxy-phenyl)-3-(1-oxo-1,3-dihydro-isoindol-2-yl)-propionamide, which is one of a class of compounds disclosed in U.S. Pat. Nos. 5,698,579; 5,877,200; 6,075,041; 6,200,987, as well as in Muller, et al., Journal of Medicinal Chemistry, 39(17), 3238-3240, 1996, and in Muller, et al., Bioorganic & Medicinal Chemistry Letters, 8, 2669-2674, 1998, each of which is incorporated herein by reference. Although this racemate offers many advantages, discovery and development continues for compounds that exhibit such desired pharmacological properties more potently, more selectively and perhaps without unwanted or toxic affects.

### 3. SUMMARY OF THE INVENTION

This invention encompasses methods of treating and preventing diseases and disorders utilizing an enantiomerically pure form of 3-(3,4-dimethoxy-phenyl)-3-(1-oxo-1,3-dihydro-isoindol-2-yl)-propionamide, referred to herein as “(+)-3-(3,4-dimethoxy-phenyl)-3-(1-oxo-1,3-dihydro-isoindol-2-yl)-propionamide,” and pharmaceutically acceptable polymorphs, salts, solvates (e.g., hydrates) and clathrates thereof. The invention further encompasses prodrugs of (+)-3-(3,4-dimethoxy-phenyl)-3-(1-oxo-1,3-dihydro-isoindol-2-yl)-propionamide and active metabolites of (+)-3-(3,4-dimethoxy-phenyl)-3-(1-oxo-1,3-dihydro-isoindol-2-yl)-propionamide as well as their use in the methods and compositions disclosed herein. Methods of this invention are useful to treat or prevent diseases, disorders or symptoms thereof while reducing or avoiding adverse effects associated with known compounds that modulate TNF-α or inhibit PDE4.

One embodiment of the invention includes methods of reducing the level of cytokines and their precursors in mammals by the administration of enantiomerically pure (+)-3-(3,4-dimethoxy-phenyl)-3-(1-oxo-1,3-dihydro-isoindol-2-yl)-propionamide.

One method of the invention is a method of treating or preventing diseases or disorders ameliorated by the inhibition of TNF-α production in mammals, which comprises administering to a patient in need thereof an effective amount of enantiomerically pure (+)-3-(3,4-dimethoxy-phenyl)-3-(1-oxo-1,3-dihydro-isoindol-2-yl)-propionamide. Such diseases or disorders include, but are not limited to, myelodysplastic syndrome; myeloproliferative syndrome; pain syndrome; macular degeneration; cancers, such as solid tumors, including, but not limited to, breast, colon, rectal, colorectal, prostate, renal, or glioma, cancers of the blood and bone marrow, such as, but not limited to, multiple myeloma, and acute and chronic leukemias (e.g., lymphoblastic, myelogenous, lymphocytic, and myelocytic leukemias); inflammatory and autoimmune diseases or disorders, including, but not limited to, rheumatoid arthritis, Crohn's disease, aphthous ulcers, erythema nodosum leprosum (ENL), cachexia, septic shock, graft versus host disease, asthma, inflammatory bowel disease (IBD), AIDS, acute respiratory distress syndrome (ARDS), chronic obstructive pulmonary diseases, dermatitis, and psoriasis.

Enantiomerically pure (+)-3-(3,4-dimethoxy-phenyl)-3-(1-oxo-1,3-dihydro-isoindol-2-yl)-propionamide and pharmaceutically acceptable prodrugs, metabolites, polymorphs, salts, solvates (e.g., hydrates) and clathrates thereof are also useful in the treatment and prevention of heart disease, such as, but not limited to, congestive heart failure, cardiomyopathy, pulmonary edema, endotoxin-mediated septic shock, acute viral myocarditis, cardiac allograft rejection, and myocardial infarction.

Another embodiment encompasses the use of enantiomerically pure (+)-3-(3,4-dimethoxy-phenyl)-3-(1-oxo-1,3-dihydro-isoindol-2-yl)-propionamide or pharmaceutically acceptable prodrugs, metabolites, polymorphs, salts, solvates (e.g., hydrates) and clathrates thereof to treat or prevent diseases or disorders ameliorated by the inhibition of PDE4. For example, the compounds or the invention or compositions thereof may be used to treat or prevent viral, genetic, inflammatory, allergic, and autoimmune diseases. Examples of such diseases include, but are not limited to: HIV; hepatitis; respiratory diseases; adult respiratory distress syndrome; bone-resorption diseases; chronic obstructive pulmonary diseases; chronic pulmonary inflammatory diseases; dermatitis; cystic fibrosis; septic shock; sepsis; endotoxic shock; hemodynamic shock; sepsis syndrome; post ischemic reperfusion injury; meningitis; psoriasis; fibrotic disease; cachexia; graft rejection including graft versus host disease; auto-immune disease; rheumatoid spondylitis; arthritic conditions, such as rheumatoid arthritis and osteoarthritis; osteoporosis; inflammatory-bowel disease; Crohn's disease; ulcerative colitis; multiple sclerosis; systemic lupus erythrematosus; ENL; radiation damage; asthma; and hyperoxic alveolar injury.

Enantiomerically pure (+)-3-(3,4-dimethoxy-phenyl)-3-(1-oxo-1,3-dihydro-isoindol-2-yl)-propionamide and pharmaceutically acceptable prodrugs, metabolites, polymorphs, salts, solvates (e.g., hydrates) and clathrates thereof are also utilized in a method of treating or preventing bacterial infections or the symptoms of bacterial infections including, but not limited to, malaria, mycobacterial infection, and opportunistic infections resulting from HIV.

The invention further encompasses methods of using enantiomerically pure (+)-3-(3,4-dimethoxy-phenyl)-3-(1-oxo-1,3-dihydro-isoindol-2-yl)-propionamide in combination with one or more additional therapeutic agents depending upon the disease or disorder to be treated as described in more detail below.

The invention further encompasses pharmaceutical compositions and single unit dosage forms comprising enantiomerically pure (+)-3-(3,4-dimethoxy-phenyl)-3-(1-oxo-1,3-dihydro-isoindol-2-yl)-propionamide or pharmaceutically acceptable prodrugs, metabolites, polymorphs, salts, solvates (e.g., hydrates) and clathrates thereof. The invention also includes kits comprising a unit dosage form of enantiomerically pure (+)-3-(3,4-dimethoxy-phenyl)-3-(1-oxo-1,3-dihydro-isoindol-2-yl)-propionamide or pharmaceutically acceptable prodrugs, metabolites, polymorphs, salts, solvates (e.g., hydrates) and clathrates thereof.

This invention particularly relates to the (+) enantiomer of 3-(3,4-dimethoxy-phenyl)-3-(1-oxo-1,3-dihydro-isoindol-2-yl)-propionamide. This compound is believed to have different pharmacological characertistics (e.g., potency and adverse effects) and other benefits as compared to racemic 3-(3,4-dimethoxy-phenyl)-3-(1-oxo-1,3-dihydro-isoindol-2-yl)-propionamide. In particular, (+)-3-(3,4-dimethoxy-phenyl)-3-(1-oxo-1,3-dihydro-isoindol-2-yl)-propionamide is believed to induce fewer or less severe adverse effects in patients as compared to racemic 3-(3,4-dimethoxy-phenyl)-3-(1-oxo-1,3-dihydro-isoindol-2-yl)-propionamide.

The invention also encompasses a method of producing enantiomerically pure (+)-3-(3,4-dimethoxy-phenyl)-3-(1-oxo-1,3-dihydro-isoindol-2-yl)-propionamide, which comprises contacting methyl 3-amino-3-(3,4-dimethoxyphenyl)-propionate with a chiral amino acid; contacting a chiral amino acid salt of (S)-methyl 3-amino-3-(3,4-dimethoxyphenyl)-propionate with methylene chloride and tetrahydrofuran or other appropriate solvents under conditions sufficient to isolate (S)-3-amino-3-(3,4-dimethoxyphenyl)-propionic acid or its salts; contacting (S)-3-amino-3-(3,4-dimethoxyphenyl)-propionic acid with phthalic dicarboxaldehyde; and contacting (S)-3-(3,4-dimethoxyphenyl)-3-(1-oxo-1,3-dihydro-isoindol-2-yl)-propionic acid with an activating agent followed by concentrated aqueous NH₃.

The invention further encompasses chiral salts of (S)-methyl 3-amino-3-(3,4-dimethoxyphenyl)-propionate.

### 3.1 Definitions

As used herein, term “Compound A” refers to enantiomerically pure (+)-3-(3,4-dimethoxy-phenyl)-3-(1-oxo-1,3-dihydro-isoindol-2-yl)-propionamide, which comes off of an HPLC column at about 18.5 minutes when that column is a 150 mm×4.6 mm Daicel Chiralpak AD column, the eluent is 20:80 IPA:hexane, and the observation wavelength is 240 nm. The ¹H NMR spectrum of (+)-3-(3,4-dimethoxy-phenyl)-3-(1-oxo-1,3-dihydro-isoindol-2-yl)-propionamide is substantially the following: δ (DMSO-d₆): 7.44-7.69 (m, 5H), 6.86-6.94 (m, 4H), 5.75 (appt. t, 1H), 4.56 (d, 1H), 4.15 (d, 1H), 3.74 (s, 3H), 3.72 (s, 3H), 2.82-3.01 (m, 2H). The ¹³C NMR spectrum of (+)-3-(3,4-dimethoxy-phenyl)-3-(1-oxo-1,3-dihydro-isoindol-2-yl)-propionamide is substantially the following: δ (DMSO-d₆): 171.27, 166.83, 148.66, 148.18, 141.69, 132.29, 131.25, 127.81, 123.42, 122.78, 119.11, 111.73, 111.07, 55.48, 51.45, 46.25, 37.93. (+)-3-(3,4-Dimethoxy-phenyl)-3-(1-oxo-1,3-dihydro-isoindol-2-yl)-propionamide, dissolved in methanol, rotates plane polarized light in the (+) direction.

(+)-3-(3,4-Dimethoxy-phenyl)-3-(1-oxo-1,3-dihydro-isoindol-2-yl)-propionamide is believed to be (S)-3-(3,4-dimethoxy-phenyl)-3-(1-oxo-1,3-dihydro-isoindol-2-yl)-propionamide, which has the following structure:

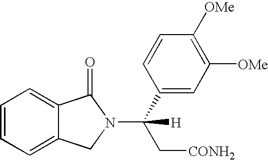

As used herein, the term “patient” refers to a mammal, particularly a human.

As used herein, the term “pharmaceutically acceptable salts” refers to salts prepared from pharmaceutically acceptable non-toxic acids or bases including inorganic acids and bases and organic acids and bases. Suitable pharmaceutically acceptable base addition salts for the compound of the present invention include metallic salts made from aluminum, calcium, lithium, magnesium, potassium, sodium and zinc or organic salts made from lysine, N,N*-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of (+)-3-(3,4-dimethoxy-phenyl)-3-(1-oxo-1,3-dihydro-isoindol-2-yl)-propionamide that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985). Prodrugs of (+)-3-(3,4-dimethoxy-phenyl)-3-(1-oxo-1,3-dihydro-isoindol-2-yl)-propionamide do not include racemic 3-(3,4-dimethoxy-phenyl)-3-(1-oxo-1,3-dihydro-isoindol-2-yl)-propionamide.

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

In specific embodiments of the invention, the term “metabolite of (+)-3-(3,4-dimethoxy-phenyl)-3-(1-oxo-1,3-dihydro-isoindol-2-yl)-propionamide” does not encompass compounds without a stereocenter. In other embodiments, the term encompasses only enantiomerically pure metabolites of (+)-3-(3,4-dimethoxy-phenyl)-3-(1-oxo-1,3-dihydro-isoindol-2-yl)-propionamide.

As used herein and unless otherwise indicated, the term “enantiomerically pure” means a stereomerically pure composition of a compound having one chiral center.

As used herein and unless otherwise indicated, “adverse effects associated with compounds used to inhibit the production of TNF-α” includes, but is not limited to gastrointestinal, renal and hepatic toxicities, leukopenia, increases in bleeding times due to, e.g., thrombocytopenia, prolongation of gestation, nausea, vomiting, somnolence, asthenia, dizziness, extra-pyramidal symptoms, akathisia, cardiovascular disturbances, male sexual dysfunction, and elevated serum liver enzyme levels. The term “gastrointestinal toxicities” includes, but is not limited to, gastric and intestinal ulcerations and erosions. The term “renal toxicities” includes, but is not limited to, conditions such as papillary necrosis and chronic interstitial nephritis.

As used herein and unless otherwise indicated, “adverse effects associated with compounds used as PDE4 inhibitors” include, but are not limited to, nausea, emesis, gastrointestinal discomfort, diarrhea, and vasculitis.

As used herein and unless otherwise indicated, “adverse effects associated with racemic 3-(3,4-dimethoxy-phenyl)-3-(1-oxo-1,3-dihydro-isoindol-2-yl)-propionamide” include, but are not limited to, abdominal pain. As used herein and unless otherwise indicated, terms “reduce or avoid adverse effects” and “reducing or avoiding adverse effects” mean the reduction of the severity of one or more adverse effects as defined herein.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4. DETAILED DESCRIPTION OF THE INVENTION

This invention encompasses novel methods for using, and compositions comprising enantiomically pure (+)-3-(3,4-dimethoxy-phenyl)-3-(1-oxo-1,3-dihydro-isoindol-2-yl)-propionamide, which is believed to have increased potency and/or an overall better therapeutic profile as compared to racemic 3-(3,4-dimethoxy-phenyl)-3-(1-oxo-1,3-dihydro-isoindol-2-yl)-propionamide. For example, the present invention encompasses the in vitro and in vivo use of (+)-3-(3,4-dimethoxy-phenyl)-3-(1-oxo-1,3-dihydro-isoindol-2-yl)-propionamide, and the incorporation of (+)-3-(3,4-dimethoxy-phenyl)-3-(1-oxo-1,3-dihydro-isoindol-2-yl)-propionamide into pharmaceutical compositions and single unit dosage forms useful in the treatment and prevention of a variety of diseases and disorders. Specific diseases and disorders are ameliorated by the reduction of levels of TNF-α and/or the inhibition of PDE4. Specific methods of the invention reduce or avoid adverse effects associated with compounds used to inhibit the production of TNF-α. Other specific methods of the invention reduce or avoid adverse effects associated with compounds used as PDE4 inhibitors. Still other specific methods reduce or avoid adverse effects associated with racemic 3-(3,4-dimethoxy-phenyl)-3-(1-oxo-1,3-dihydro-isoindol-2-yl)-propionamide.

Methods of the invention include methods of treating and preventing diseases and disorders including, but not limited to, solid tumor cancers, blood-born cancers, inflammatory diseases and autoimmune diseases.

Pharmaceutical and dosage forms of the invention, which comprise enantiomerically pure (+)-3-(3,4-dimethoxy-phenyl)-3-(1-oxo-1,3-dihydro-isoindol-2-yl)-propionamide or a pharmaceutically acceptable prodrug, metabolite, polymorph, salt, solvate (e.g., hydrate), or clathrate thereof, are encompassed by the invention, and can be used in its methods.

Without being limited by theory, (+)-3-(3,4-dimethoxy-phenyl)-3-(1-oxo-1,3-dihydro-isoindol-2-yl)-propionamide can inhibit TNF-α production in mammal cells. Consequently, a first embodiment of the invention relates to a method of inhibiting TNF-α production which comprises contacting a cell exhibiting abnormal TNF-α production with an effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate) and clathrate thereof. In a particular embodiment, the invention relates to a method of inhibiting TNF-α production which comprises contacting a mammalian cell exhibiting abnormal TNF-α production with an effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof.

The invention also relates to a method of treating or preventing diseases or disorders ameliorated by the reduction of TNF-α levels in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. Diseases or disorders ameliorated by reduction of TNF-α levels include, but are not limited to, diabetic retinopathy, retinopathy of prematurity, corneal graft rejection, neovascular glaucoma, retrolental fibroplasia, proliferative vitreoretinopathy, trachoma, myopia, optic pits, epidemic keratoconjunctivitis, atopic keratitis, superior limbic keratitis, pterygium keratitis sicca, sjogrens, acne rosacea, phylectenulosis, syphilis, lipid degeneration, bacterial ulcer, fungal ulcer, Herpes simplex infection, Herpes zoster infection, protozoan infection, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, sickle cell anemia, sarcoid, pseudoxanthoma elasticum, Pagets disease, vein occlusion, artery occlusion, carotid obstructive disease, chronic uveitis, chronic vitritis, Lyme's disease, Eales disease, Bechet's disease, retinitis, choroiditis, presumed ocular histoplasmosis, Bests disease, Stargarts disease, pars planitis, chronic retinal detachment, hyperviscosity syndromes, toxoplasmosis, sclerosing cholangitis, rubeosis, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, and 5q-syndrome.

A further embodiment of the invention relates to a method of treating or preventing cancer, including but not limited to, solid tumor, blood-born tumor, and multiple myeloma in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate) or clathrate thereof, in particular, wherein the patient is a mammal.

In another embodiment, the invention relates to a method of inhibiting PDE4 activity which comprises contacting PDE4 with an effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate) or clathrate thereof.

In another embodiment, the invention relates to a method of controlling cAMP levels in a cell which comprises contacting the cell with an effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. As used herein, the term “controlling cAMP levels” includes preventing and reducing the rate of the breakdown of adenosine 3′,5′-cyclic monophosphate (cAMP) in a cell or increasing the amount of adenosine 3′,5′-cyclic monophosphate present in a cell, preferably a mammalian cell, more preferably a human cell. In a particular method, the rate of cAMP breakdown is reduced by about 10, 25, 50, or 100 percent as compared to the rate in comparable cells that have not been contacted with a compound of the invention.

A further embodiment of the invention relates to a method of treating or preventing diseases or disorders ameliorated by the inhibition of PDE4 in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. Disorders ameliorated by the inhibition of PDE4 include, but are not limited to, respiratory diseases, asthma, inflammation (e.g., inflammation due to reperfusion), chronic or acute obstructive pulmonary diseases, chronic or acute pulmonary inflammatory diseases, inflammatory bowel disease, Crohn's Disease, Bechet's Disease, and colitis.

A further embodiment of the invention relates to a method of treating or preventing asthma, inflammation (e.g., contact dermatitis, atopic dermatitis, psoriais, rheumatoid arthritis, osteoarthritis, inflammatory skin disease, inflammation due to reperfusion), chronic or acute obstructive pulmonary diseases, chronic or pulmonary inflammatory diseases, inflammatory bowel disease, Crohn's Disease, Bechet's Disease or colitis in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof; in particular wherein the patient is a mammal.

Another embodiment of the invention encompasses methods of treating, managing or preventing myelodysplastic syndrome (MDS) which comprise administering to a patient in need of such treatment, management or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. A further embodiment of the invention encompasses the use of the compound in combination with a conventional therapy presently used to treat, prevent or manage MDS, such as hematopoietic growth factors, cytokines, cancer chemotherapeutics, stem cell transplantation and other transplantations.

Another embodiment of the invention encompasses methods of treating, managing or preventing myeloproliferative disease (MPD) which comprise administering to a patient in need of such treatment, management or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. A further embodiment of the invention encompasses the use of the compound in combination with conventional therapies presently used to treat, prevent or manage MPD such as, but not limited to, hydroxyurea, anagrelide, interferons, kinase inhibitors, cancer chemotherapeutics, stem cell transplanation and other transplantations.

The invention also encompasses a method of treating, preventing or managing pain including, but not limited to, complex regional pain syndrome and fibromyalgia, which comprises administering to a patient in need of such treatment, prevention or management a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. In another embodiment, the administration is before, during or after surgery or physical therapy directed at reducing or avoiding a symptom of pain including, but not limited to, complex regional pain syndrome and fibromyalgia in the patient.

The invention also encompasses a method of treating, preventing or managing macular degeneration (e.g., age-related macular degeneration), which comprises administering to a patient in need of such treatment, prevention or management a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. Yet another embodiment of the invention encompasses methods for treating or managing macular degeneration, comprising administering to a patient in need thereof an effective amount of enantiomerically pure (+)-3-(3,4-dimethoxy-phenyl)-3-(1-oxo-1,3-dihydro-isoindol-2-yl)-propionamide, or a pharmaceutically acceptable salt, solvate (e.g., hydrate), stereoisomer, clathrate, or prodrug thereof, in combination with a conventional therapy presently used to treat or manage macular degeneration such as, but not limited to, surgical intervention (e.g., laser photocoagulation therapy and photodynamic therapy).

In particular methods of the invention, (+)-3-(3,4-dimethoxy-phenyl)-3-(1-oxo-1,3-dihydro-isoindol-2-yl)-propionamide, or a pharmaceutically acceptable prodrug, metabolite, polymorth, salt, solvate (e.g., hydrate), or clathrate thereof, is adjunctively administered with at least one additional therapeutic agent. Examples of additional therapeutic agents include, but are not limited to, anti-cancer drugs, anti-inflammatories, biologics, IMiDs™, antihistamines, antibiotics, anti-virals, GM-CSF, IL-2, NSAID's, steroids and decongestants. More specifically, the invention encompasses the combined use of (+)-3-(3,4-dimethoxy-phenyl)-3-(1-oxo-1,3-dihydro-isoindol-2-yl)-propionamide with thalidomide, 4-(amino)-2-(2,6-dioxo-(3-piperidyl))-isoindoline-1,3-dione (Actimid™), 3-(4-amino-1-oxo-1,3-dihydro-isoindol-2-yl)-piperidine-2,6-dione (Revimid™), or a JNK inhibitor, as discussed in more detail below.

### 4.1 Synthesis and Preparation

Racemic 3-(3,4-dimethoxy-phenyl)-3-(1-oxo-1,3-dihydro-isoindol-2-yl)-propionamide is readily prepared according to the methods in U.S. Pat. No. 5,698,579, the entirety of which is incorporated herein by reference.

(+)-3-(3,4-Dimethoxy-phenyl)-3-(1-oxo-1,3-dihydro-isoindol-2-yl)-propionamide can be isolated from the racemic compound by techniques known in the art. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

(+)-3-(3,4-Dimethoxy-phenyl)-3-(1-oxo-1,3-dihydro-isoindol-2-yl)-propionamide can also be prepared from (S)-3-(3,4-dimethoxyphenyl)-3-(1-oxo-1,3-dihydro-isoindol-2-yl)-propionic acid, which is obtained from, for example, (S)-3-amino-3-(3,4-dimethoxyphenyl)-propionic acid and phthalic dicarboxaldehyde in acetic acid. (See, e.g., Example 2 herein).

### 4.2 Methods of Treatment and Prevention

The invention encompasses methods of treating and preventing diseases or disorders ameliorated by the reduction of TNF-α levels in a patient which comprise administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof. Diseases or disorders ameliorated by the reduction of TNF-α levels include, but are not limited to:

myelodysplastic syndrome, myeloproliferative syndrome, pain (e.g.,complex regional pain syndrome and fibromyalgia) and macular degeneration;

heart disease, such as congestive heart failure, cardiomyopathy, pulmonary edema, endotoxin-mediated septic shock, acute viral myocarditis, cardiac allograft rejection, and myocardial infarction;

solid tumors, including but not limited to, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, and retinoblastoma;

blood-born tumors including but not limited to, acute lymphoblastic leukemia (ALL), acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma and acute and chronic leukemias, for example, lymphoblastic, myelogenous, lymphocytic, and myelocytic leukemias; and

diabetic retinopathy, retinopathy of prematurity, corneal graft rejection, neovascular glaucoma, retrolental fibroplasia, proliferative vitreoretinopathy, trachoma, myopia, optic pits, epidemic keratoconjunctivitis, atopic keratitis, superior limbic keratitis, pterygium keratitis sicca, sjogrens, acne rosacea, phylectenulosis, syphilis, lipid degeneration, bacterial ulcer, fungal ulcer, Herpes simplex infection, Herpes zoster infection, protozoan infection, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, sickle cell anemia, sarcoid, pseudoxanthoma elasticum, Pagets disease, vein occlusion, artery occlusion, carotid obstructive disease, chronic uveitis, chronic vitritis, Lyme's disease, Eales disease, Bechet's disease, retinitis, choroiditis, presumed ocular histoplasmosis, Bests disease, Stargarts disease, pars planitis, chronic retinal detachment, hyperviscosity syndromes, toxoplasmosis, sclerosing cholangitis, rubeosis, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, and 5q-syndrome.

Specific methods of the invention further comprise the administration of an additional therapeutic agent (i.e., a therapeutic agent other than (+)-3-(3,4-dimethoxy-phenyl)-3-(1-oxo-1,3-dihydro-isoindol-2-yl)-propionamide). Examples of additional therapeutic agents include, but are not limited to, anti-cancer drugs such as, but are not limited to: alkylating agents, nitrogen mustards, ethylenimines, methylmelamines, alkyl sulfonates, nitrosoureas, triazenes, folic acid analogs, pyrimidine analogs, purine analogs, vinca alkaloids, epipodophyllotoxins, antibiotics, topoisomerase inhibitors, JNK (C-Jun Kinase) inhibitors, IMiDs™ (Celgene Corporation, N.J.), and anti-cancer vaccines. Specific JNK inhibitors are disclosed in U.S. patent application Ser. Nos. 09/642,557, 09/910,950, 10/414,839, 10/004,645 and 10/071,390, the entireties of which are incorporated herein by reference. Specific IMiDs™ are disclosed in U.S. patent application Ser. No. 10/438,213 filed on May 15, 2003, and U.S. Pat. Nos. 6,281,230, 5,635,517, 5,798,368, 6,395,754, 5,955,476, 6,403,613, 6,380,239 and 6,458,810, the entireties of which are incorporated herein by reference.

Specific additional therapeutic agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; Actimid™ (4-(amino)-2-(2,6-dioxo-(3-piperidyl))-isoindoline-1,3-dione); azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; Erbitux™, esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; Revimid™ (3-(4-amino-1-oxo-1,3-dihydro-isoindol-2-yl)-piperidine-2,6-dione); riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thalidomide; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors including antibodies; antagonist D; antagonist G; antarelix; anti-dorsalizing morphogenetic protein-l; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride, pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The invention further encompasses a method of treating or preventing diseases or disorders ameliorated by the inhibition of PDE4 in a patient which comprises administering to a patient in need of such treatment or prevention a therapeutically or prophylactically effective amount of (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof.

Disorders ameliorated by the inhibition of PDE4 include, but are not limited to, asthma, inflammation, chronic or acute obstructive pulmonary disease, chronic or acute pulmonary inflammatory disease, inflammatory bowel disease, Crohn's disease, ulcerative colitis, Bechet's Disease, HSP, and inflammation due to reperfusion.

Specific methods of the invention can comprise the administration of an additional therapeutic agent such as, but not limited to, anti-inflammatory drugs, antihistamines and decongestants. Examples of such additional therapeutic agents include, but are not limited to: antihistamines including, but not limited to, ethanolamines, ethylenediamines, piperazines, and phenothiazines; anti-inflammatory drugs; non-steroidal anti-inflammatory drugs (NSAIDS), including, but not limited to, salicylates, acetaminophen, indomethacin, sulindac, etodolac, fenamates, tolmetin, ketorolac, diclofenac, ibuprofen, naproxen, fenoprofen, ketoprofen, flurbiprofen, oxaprozin, piroxicam, meloxicam, pyrazolon derivatives; specific cyclooxygenase-2 inhibitors including, but not limited to, celecoxib, rofecoxib, and valdecoxib; disease modifying antirheumatic drugs including, but not limited to, methotrexate, sulfasalasine, and injectable gold; immunosuppressants including, but not limited to, leflunomide, pimecrolimus, azathioprine, cyclosporin, penicillamine, and 6-mercaptopurine; topical retinoids including, but not limited to, tazarotene; vitamin D analogs including, but not limited to, calcipotriene; biological anti-inflammatory agents including, but not limited to, etanercept, infliximab, anakinra, efalizumab, and omalizumab; beta-2 adrenergic receptor agonists including, but not limited to, albuterol and salmeterol; anti-cholinergics including, but not limited to, ipratropium; steroids including, but not limited to, cortical steroids and adrenocortical steroids such as prednisone, methylprednisone, hydrocortisone, budesonide, betamethasone, and dexamethasone; and combination therapies including, but not limited to, beta-2 adrenergic agonists plus steroids or beta-2 adrenergic agonists plus anti-cholinergics.

Active compounds of the invention (e.g., (+)-3-(3,4-dimethoxy-phenyl)-3-(1-oxo-1,3-dihydro-isoindol-2-yl)-propionamide) may be used in the treatment or prevention of a wide range of diseases and conditions. The magnitude of a prophylactic or therapeutic dose of a particular active agent of the invention in the acute or chronic management of a disease or condition will vary, however, with the nature and severity of the disease or condition, and the route by which the active agent is administered. The dose, and perhaps the dose frequency will also vary according to the age, body weight, and response of the individual patient. Suitable dosing regimens can be readily selected by those skilled in the art with due consideration of such factors. In one embodiment of the invention, the recommended daily dose range for the conditions described herein is from about 1 mg to about 10,000 mg per day, given as a single once-a-day dose, or preferably in divided doses throughout a day. The daily dose can be administered twice daily in equally divided doses. Specific daily dose ranges are from about 1 mg to about 5,000 mg per day, from about 10 mg to about 2,500 mg per day, from about 100 mg to about 800 mg per day, from about 100 mg to about 1,200 mg per day, or from about 25 mg to about 2,500 mg per day. In managing the patient, the therapy should be initiated at a lower dose, perhaps from about 1 mg to about 25 mg, and increased if necessary up to about 200 mg to about 1,200 mg per day as either a single dose or divided doses, depending on the patient's global response.

It may be necessary to use dosages of the active agent outside the ranges disclosed herein in some cases, as will be apparent to those of ordinary skill in the art. The clinician or treating physician will know how and when to interrupt, adjust, or terminate therapy in conjunction with individual patient response.

The phrases “therapeutically effective amount,” “prophylactically effective amount” and “therapeutically or prophylactically effective amount,” as used herein, encompass the above described dosage amounts and dose frequency schedules. Different therapeutically effective amounts may be applicable for different diseases and conditions, as will be readily known by those of ordinary skill in the art. Similarly, amounts sufficient to treat or prevent such disorders, but insufficient to cause, or sufficient to reduce, adverse effects associated with conventional therapies are also encompassed by the above described dosage amounts and dose frequency schedules.

### 4.3 Pharmaceutical Compositions

Pharmaceutical compositions and single unit dosage forms comprising enantiomerically pure (+)-3-(3,4-dimethoxy-phenyl)-3-(1-oxo-1,3-dihydro-isoindol-2-yl)-propionamide, or a pharmaceutically acceptable metabolite, polymorph, prodrug, salt, solvate (e.g., hydrate), or clathrate thereof, are also encompassed by the invention. Individual dosage forms of the invention may be suitable for oral, mucosal (including rectal, nasal, or vaginal), parenteral (including subcutaneous, intramuscular, bolus injection, intraarterial, or intravenous), sublingual, transdermal, buccal, or topical administration.

Typical pharmaceutical compositions and dosage forms of the invention comprise enantiomerically pure (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof, and one or more pharmaceutically acceptable excipients. A particular pharmaceutical composition comprises enantiomerically pure (+)-3-(3,4-dimethoxy-phenyl)-3-(1-oxo-1,3-dihydro-isoindol-2-yl)-propionamide, or a pharmaceutically acceptable metabolite, polymorph, prodrug, salt, solvate (e.g., hydrate), or clathrate thereof, and at least one additional therapeutic agent. Examples of additional therapeutic agents include, but are not limited to, anti-cancer drugs and anti-inflammation therapies including, but not limited to, those listed above in section 4.2.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic solutions), or transdermal administration to a patient. Examples of dosage forms include, but are not limited to: tablets; caplets; capsules, such as soft elastic gelatin capsules; cachets; troches; lozenges; dispersions; suppositories; eye drops; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active agents it comprises than a dosage form used in the chronic treatment of the same disease. Similarly, a parenteral dosage form may contain smaller amounts of one or more of the active agents it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more carriers or excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active agents in the dosage form.

Lactose-free compositions of the invention can comprise excipients that are well known in the art and are listed, for example, in the U.S. Pharmocopia (USP) SP (XXI)/NF (XVI). In general, lactose-free compositions comprise active agents, a binder/filler, and a lubricant in pharmaceutically compatible and pharmaceutically acceptable amounts. Preferred lactose-free dosage forms comprise active agents, microcrystalline cellulose, pre-gelatinized starch, and magnesium stearate.

This invention further encompasses anhydrous pharmaceutical compositions and dosage forms comprising active agents, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing agents and low moisture or low humidity conditions. Pharmaceutical compositions and dosage forms that comprise lactose and at least one active agent that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active agent will decompose. Such compounds, which are referred to herein as “stabilizers,” include, but are not limited to, antioxidants such as ascorbic acid, pH buffers, or salt buffers.

Like the amounts and types of excipients, the amounts and specific types of active agents in a dosage form may differ depending on factors such as, but not limited to, the route by which it is to be administered to patients. However, typical dosage forms of the invention comprise (+)-3-(3,4-dimethoxy-phenyl)-3-(1-oxo-1,3-dihydro-isoindol-2-yl)-propionamide, or a pharmaceutically acceptable prodrug, metabolite, polymorph, salt, solvate (e.g., hydrate), or clathrate thereof having 1 mg, 5 mg, 10 mg, 50 mg, 75 mg, 100 mg, 250 mg, 500 mg and 750 mg of active agent. More specifically, the invention encompasses solid oral dosage form in these unit dose amounts. Similarly, solid injectable (optionally lyophilized) dosage forms in similar unit dosage amounts are encompassed by the invention.

### 4.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active agents, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active agent(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active agents with liquid carriers, finely divided solid carriers, or both, and then shaping the product into the desired presentation if necessary.

For example, a tablet can be prepared by compression or molding. Compressed tablets can be prepared by compressing in a suitable machine the active agents in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active agents should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### 4.3.2 Controlled/Delayed Release Dosage Forms

Active agents of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active agents using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active agent) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active agent can be stimulated by various conditions including, but not limited to, pH, temperature, enzymes, water, or other physiological conditions or compounds.

### 4.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active agents disclosed herein can also be incorporated into the parenteral dosage forms of the invention.

### 4.3.4 Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active agents.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional agents are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active agents of the invention. For example, penetration enhancers can be used to assist in delivering the active agents to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active agents. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active agents so as to improve delivery. In this regard, stearates can serve as a lipid vehicle for the formulation, as an emulsifying agent or surfactant, and as a delivery-enhancing or penetration-enhancing agent. Different salts, hydrates or solvates of the active agents can be used to further adjust the properties of the resulting composition.

### 4.3.5 Kits

Typically, active agents of the invention are preferably not administered to a patient at the same time or by the same route of administration. This invention therefore encompasses kits which, when used by the medical practitioner, can simplify the administration of appropriate amounts of active agents to a patient.

A typical kit of the invention comprises a unit dosage form of (+)-3-(3,4-dimethoxy-phenyl)-3-(1-oxo-1,3-dihydro-isoindol-2-yl)-propionamide, or a pharmaceutically acceptable salt, solvate (e.g., hydrate), clathrate, polymorph or prodrug thereof, and a unit dosage form of a second active agent. Examples of second active agents include, but are not limited to, those listed in section 4.2 above.

Kits of the invention can further comprise devices that are used to administer the active agents. Examples of such devices include, but are not limited to, syringes, drip bags, patches, and inhalers.

Kits of the invention can further comprise pharmaceutically acceptable vehicles that can be used to administer one or more active agents. For example, if an active agent is provided in a solid form that must be reconstituted for parenteral administration, the kit can comprise a sealed container of a suitable vehicle in which the active agent can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

### 5.1 Example 1

### Synthesis of Racemic 3-(3,4-DIMETHOXY-PHENYL)-3-(1-OXO-1,3-DIHYDRO-ISOINDOL-2-YL)-PROPIONAMIDE

To a stirred solution of 3-(3,4-dimethoxyphenyl)-3-(1-oxoisoindolin-2-yl)propionic acid (917 mg, 2.70 mmol) in 15 mL of tetrahydrofuran under nitrogen was added carbonyldiimidazole (438 mg, 2.70 mmol), and a few crystals of 4-N,N-dimethylaminopyridine [DMAP]. The reaction mixture was stirred for 1.5 hours and then 0.25 mL of 15N ammonium hydroxide was added. After 20 minutes, the reaction mixture was concentrated in vacuo and the residue slurried in water. The resulting solid was isolated by filtration and dried in vacuo to afford 0.58 g (80%) of crude product as an off-white powder. The crude product did not precipitate from water immediately. The product crystallized from aqueous solution upon sitting for several days after an ether wash to afford 0.26 g (22%) of 3-(3,4-dimethoxyphenyl)-3-(1-oxoisoindolin-2-yl)propionamide as white needles: ¹H NMR (DMSO-₆, 250 MHz) 7.8-7.4 (m, 5H), 7.1-6.85 (m, 4H), 5.76 (m, 1H), 4.57 (d, 17.6 Hz, 1H), 4.15 (d, J=17.6 Hz, 1H), 3.74 (s, 3H), 3.72 (s, 3H), 3.1-2.8 (m, 2H); ¹³C NMR (DMSO-d₆), 171.2, 166.8, 148.6, 148.1, 141.6, 132.2, 132.2, 131.2, 127.8, 123.4, 122.7, 119.0, 111.6, 111.0, 55.4, 51.4, 46.2, 37.9; Anal. Calcd for C₁₉H₂ON₂O₄: Theory C, 67.05; H, 5.92; N, 8.23. Found: C, 66.74; H, 5.88; N, 8.02.

### 5.2 Example 2

### Synthesis of (+)-3-(3,4-DIMETHOXY-PHENYL)-3-(1-OXO-1,3-DIHYDRO-ISOINDOL-2-YL)-PROPIONAMIDE

### Preparation of 3-amino-3-(3,4-dimethoxyphenyl)propionic acid

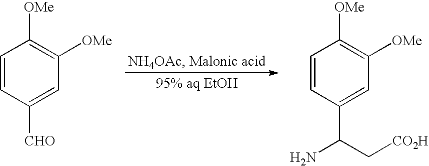

A 2 L 3-necked round bottom flask equipped with a mechanical stirrer and thermometer was charged with 3,4-dimethoxybenzaldehyde (194.5 g, 1.17 mol), ammonium acetate (180.4 g, 2.34 mol) and 600 mL of 95% aqueous ethanol. The stirred slurry was heated to 45° C., yielding a brown solution to which was added malonic acid (121.8 g, 1.17 mol). The resulting thick slurry was heated to reflux and held at reflux for 16 hours. The stirred mixture was then allowed to cool to ambient temperature. The slurry was filtered and the filter cake was washed with 300 mL of cold (˜5° C.) ethanol. The solid was dried in vacuo at 60° C. to a constant weight, affording 147.6 g (56% yield) of the product as a white powder.

### Preparation of methyl 3-amino-3-(3,4-dimethoxyphenyl)propionate hydrochloride salt

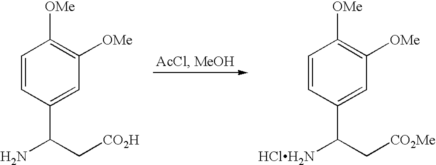

A 2 L 3-necked round bottom flask equipped with a mechanical stirrer, thermometer, and a dropping funnel was charged with 3-amino-3-(3,4-dimethoxyphenyl)propionic acid (129.8 g, 0.576 mol) and 780 mL of methanol. This stirred slurry was cooled to 0° C. and charged with acetyl chloride over 20 minutes while the reaction temperature was maintained between 0° C. and 4° C. The stirring was continued for 20 minutes at 0° C. and overnight at ambient temperature. The reaction mixture was concentrated to about 2 volumes to which was added 520 mL of methyl tertiary-butyl ether (MTBE). The resulting slurry was stirred at ambient temperature for 2 hours. The slurry was then filtered and the filter cake was washed with MTBE (260 mL). The solid was dried in vacuo at 55° C. to a constant weight, affording 145.8 g (92% yield) of the product as a white crystalline solid. HPLC (10/90 CH₃CN/0.1% aqueous H₃PO₄, Waters Nova-Pak C18 Column, 3.9×150 mm, 4 μm, 1.0 mL/min., 210 nm): RT 4.63 min. (>99.0% by area). ¹H NMR (DMSO-d₆) δ: 8.71 (brs, 3H), 7.31 (d, 1H), 6.93-7.04 (m, 2H), 4.51 (appt. t, 1H), 3.77 (s, 3H), 3.75 (s, 3H), 3.56 (s, 3H), 3.15-3.24 (dd, 1H), 2.94-3.04 (dd, 1H).

### Preparation of methyl 3-amino-3-(3,4-dimethoxyphenyl)propionate

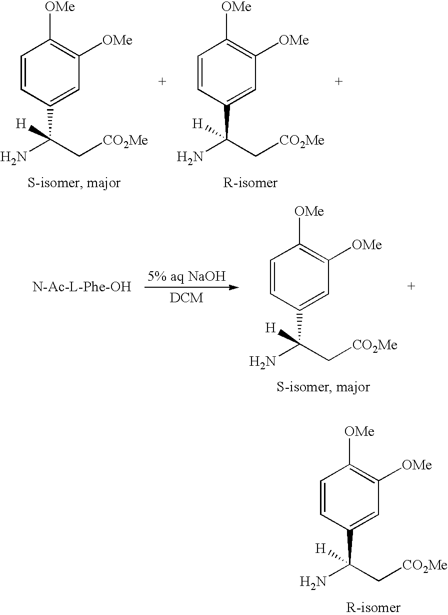

A 3 L 3-necked round bottom flask equipped with a mechanical stirrer, thermometer, and a dropping funnel was charged with the methyl 3-amino-3-(3,4-dimethoxyphenyl)-propionate N-acetyl-L-leucine/free amine mixture (mother liquor from the above step, 56% ee S-isomer), methylene chloride (820 mL), and city water (410 mL). This mixture was stirred and the resulting slurry was cooled to 0° C. and charged with 5% aq NaOH (to pH 11-12, 100 mL) over 1 hour, while the reaction temperature was maintained at about 0° C. After the addition of the aq NaOH was complete, stirring was continued for 5 minutes. The organic layer was separated and the aqueous part was extracted with methylene chloride (220 mL×2). The methylene chloride parts were combined and washed with water (210 mL×2). The methylene chloride solution was concentrated on a rotovap while maintaining the bath temperature below 25° C., generating 74.2 g (98% yield) of the crude methyl 3-amino-3-(3,4-dimethoxyphenyl)propionate as a colorless oil with 57% ee. The crude free base was used in the next step without further purification. Chiral HPLC (10/90 MeOH/aq HClO₄ @pH 1.0, Daicel Crownpak CR (+) column, 4×150 mm, 5 μm, 0.7 mL/min., 240 nm): 22.27 min. (R-isomer, 21.7% by area), 27.80 min. (S-isomer, 78.3% by area). ¹H NMR (DMSO-d₆) δ: 6.99 (s, 1H), 6.85 (s, 2H), 4.14 (appt. t, 1H), 3.74 (s, 3H), 3.71 (s, 3H), 3.56 (s, 3H), 2.55 (d, 2H), 1.91 (brs, 2H).

### Preparation of (S)-Methyl 3-amino-3-(3,4-dimethoxyphenyl)-propionate N-acetyl-D-phenylalanine salt

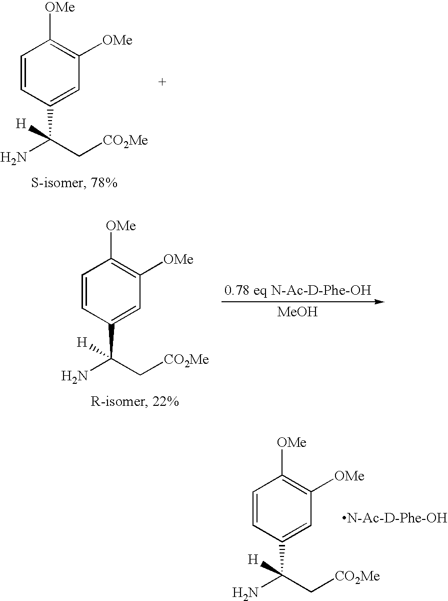

A 2 L 3-necked round bottom flask equipped with a mechanical stirrer, thermometer, and condenser was charged with S-enriched methyl 3-amino-3-(3,4-dimethoxyphenyl)-propionate (70.9 g, 0.296 mol), N-acetyl-D-phenylalanine (47.9, 0.231 mol), and methanol (1,060 mL). The stirred slurry was heated to reflux and held at reflux for 1 hour. The stirred mixture was allowed to cool to ambient temperature and stirring was continued for another 3 hours at ambient temperature. The slurry was filtered and the filter cake was rinsed with methanol (290 mL). The solid was air-dried and then dried in vacuo at 55° C. to a constant weight, affording 85.0 g (82% yield) of (S)-methyl 3-amino-3-(3,4-dimethoxyphenyl)-propionate N-acetyl-D-phenylalanine salt (90.7% ee). Chiral HPLC (10/90 MeOH/aq HCl₄ @pH 1.0, Daicel Crownpak CR (+) column, 4×150 mm, 5 μm, 0.7 mL/min., 240 nm): 22.5 min. (R-isomer, 4.5% by area), 27.15 min. (S-isomer, 92.7% by area). ¹H NMR (DMSO-d₆) δ: 7.88 (d, 1H), 7.14-7.27 (m, 5H), 7.07 (s, 1H), 6.89 (s, 2H), 4.25-4.33 (m, 2H), 3.74 (s, 3H), 3.72 (s, 3H), 3.56 (s, 3H), 3.01-3.09 (dd, 1H), 2.66-2.86 (m, 3H), 1.76 (s, 3H).

### Generation of (S)-Methyl 3-amino-3-(3,4-dimethoxyphenyl)propionate

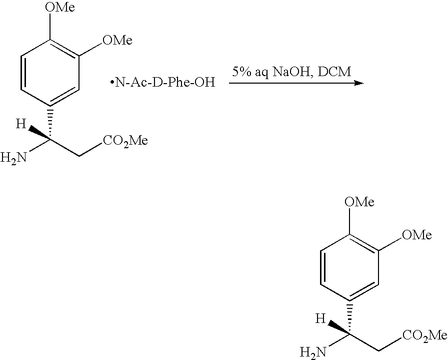

A 2 L 3-necked round bottom flask equipped with a mechanical stirrer, thermometer, and a dropping funnel was charged with (S)-methyl 3-amino-3-(3,4-dimethoxyphenyl)-propionate N-acetyl-D-phenylalanine salt (84.8 g, 0.190 mol), methylene chloride (510 mL), and city water (250 mL). This stirred slurry was cooled to 0° C. and charged with 5% aq NaOH (154 mL, to pH 11-12) over 2.5 hours, the reaction temperature was maintained at ˜0° C. After completion of the addition of the aq NaOH, stirring was continued for another 5 minutes. The organic layer was separated and the aqueous part was extracted with methylene chloride (130 mL×2). The methylene chloride parts were combined and washed with water (130 mL×2). The methylene chloride solution was concentrated on a rotovap while maintaining the bath temperature below 25° C., generating 45.6 g (quantitative yield) of (S)-methyl 3-amino-3-(3,4-dimethoxyphenyl)propionate (91.9% ee) as a colorless oil. Chiral HPLC (10/90 MeOH/aq HClO₄ @pH 1.0, Daicel Crownpak CR (+) column, 4×150 mm, 5 μm, 0.7 mL/min., 240 nm): 23.06 min. (R-isomer, 3.8% by area), 28.14 min. (S-isomer, 90.2% by area). HPLC (10/90 CH₃CN/0.1% aq H₃PO₄, Waters Nova-Pak C18 Column, 3.9×150 mm, 4 μm, 1.0 mL/min., 210 nm): RT 4.68 min. (>99.0% by area). ¹H NMR (DMSO-d₆) δ: 6.99 (s, 1H), 6.85 (s, 2H), 4.14 (brs, 1H), 3.74 (s, 3H), 3.71 (s, 3H), 3.56 (s, 3H), 2.55 (d, 2H), 1.90 (brs, 2H).

### Synthesis of (S)-3-Amino-3-(3,4-dimethoxyphenyl)propionic acid

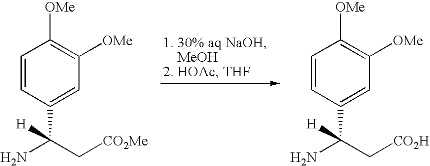

A 1 L 3-necked round bottom flask equipped with a mechanical stirrer, thermometer, and a dropping funnel was charged with the crude (S)-methyl 3-amino-3-(3,4-dimethoxyphenyl)propionate (45.4 g, 0.190 mol) from the above step and methanol (230 mL). To the stirred solution was charged 30% aq NaOH (38 mL) over 15 minutes while maintaining reaction temperature below 25° C. The resulting mixture was then stirred at ambient temperature for another 2 hours. The reaction mixture was then concentrated on a rotovap with the bath temperature controlled below 30° C., yielding a thick oil. About 110 mL distillate was collected. This oil was diluted with THF (450 mL) and stirred. To this stirred solution was added dropwise acetic acid (45 mL) with the reaction temperature kept below 25° C. The resulting slurry was stirred at ambient temperature for 1.5 hours. The slurry was then filtered and the filter cake was washed with THF (180 mL). The solid was dried in vacuo at 55° C. overnight, affording 60.5 g (140% yield) of crude white (S)-3-amino-3-(3,4-dimethoxyphenyl)propionic acid that was used in the next step without further purification. HPLC (10/90 CH₃CN/0.1% aq H₃PO₄, Waters Nova-Pak C18 Column, 3.9×150 mm, 4 μm, 1.0 mL/min., 210 nm): RT 2.22 min. (>99.0% by area). ¹H NMR (D₂O) δ: 6.85-6.92 (m, 3H), 4.42 (appt. t, 1H), 3.70 (s, 1H), 3.69 (s, 3H), 2.57-2.78 (m, 2H), 1.73 (s, 4H).

### Synthesis (S)-3-(3,4-Dimethoxyphenyl)-3-(1-oxo-1,3-dihydro-isoindol-2-yl)propionic acid

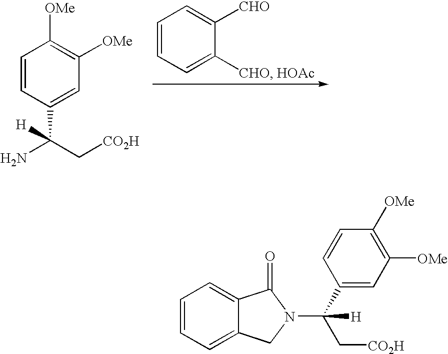

A 2 L 3-necked round bottom flask equipped with a mechanical stirrer, thermometer, and condenser was charged with the crude product from (S)-3-amino-3-(3,4-dimethoxyphenyl)propionic acid (60.5 g, 0.190 mol, from the above step), phthalic dicarboxaldehyde (25.5 g, 0.190 mol), and acetic acid (450 mL). The slurry was stirred at ambient temperature for 2 hours, generating a light brown solution. The stirred solution was heated to reflux and held at reflux for 45 minutes. The reaction mixture was then concentrated to a thick oil. About 360 mL of distillate was collected. The resulting mixture was diluted with city water (100 mL) followed by addition of MTBE (220 mL) and another portion of city water (350 mL). The resulting slurry was stirred vigorously at ambient temperature for 2 hours. The slurry was filtered and the filter cake was washed with city water (90 mL) and MTBE (90 mL×2). The solid was air-dried and then dried in vacuo at 55° C. to a constant weight, affording 54.5 g of an off-white (S)-3-(3,4-dimethoxyphenyl)-3-(1-oxo-1,3-dihydro-isoindol-2-yl)propionic acid [overall 84% yield from (S)-methyl 3-amino-3-(3,4-dimethoxyphenyl)propionate N-acetyl-D-phenylalanine salt]. HPLC (45/55 CH³CN/0.1% aq H₃PO₄, Waters Nova-Pak C18 Column, 3.9×150 mm, 4 μM, 1.0 mL/min., 210 nm): RT 1.86 min. (>99.0% by area). ¹H NMR (DMSO-d₆) δ: 12.37 (s, 1H), 7.45-7.70 (m, 4H), 6.99 (s, 1H), 6.93 (s, 2H), 5.72 (appt. t, 1H), 4.51 (d, 1H), 4.12 (d, 1H), 3.75 (s, 3H), 3.73 (s, 3H), 3.05-3.22 (m, 2H).

### Preparation of (+)-3-(3,4-Dimethoxy-phenyl)-3-(1-oxo-1,3-dihydro-isoindol-2-yl)-propionamide

Assuming, without being limited by theory, that (+)-3-(3,4-dimethoxy-phenyl)-3-(1-oxo-1,3-dihydro-isoindol-2-yl)-propionamide is (S)-3-(3,4-dimethoxy-phenyl)-3-(1-oxo-1,3-dihydro-isoindol-2-yl)-propionamide, this step is represented below:

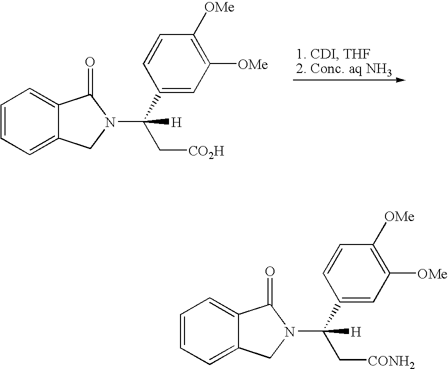

A 1 L 3-necked round bottom flask equipped with a mechanical stirrer and thermometer was charged with (S)-3-(3,4-dimethoxyphenyl)-3-(1-oxo-1,3-dihydro-isoindol-2-yl)-propionic acid (36.2 g, 0.106 mol), THF (250 mL), and CDI (19.0 g, 117 mmol). The reaction mixture was then stirred at ambient temperature for 2 hours. The resulting mixture was charged with conc. aq NH₃ over 15 minutes while the reaction temperature maintained between 15° C. and 20° C. The reaction mixture was then allowed to warm to ambient temperature and stirred at ambient temperature for another 1 hour. The reaction mixture was diluted with DI water (110 mL) and concentrated on a rotovap to generate about 250 mL of distillate. The resulting mixture was charged with another portion of DI water (250 mL) and concentrated to generate about 70 mL of distillate. The resulting slurry was filtered and the filter cake was washed with DI water (110 mL×3). The solid was air-dried and then dried in vacuo at 55° C. to a constant weight, affording 33.4 g (92% yield) of an off-white (R)-3-(3,4-dimethoxyphenyl)-3-(1-oxo-1,3-dihydro-isoindol-2-yl)propionamide with >99.5% ee. Mp 191-193° C. Chiral HPLC (20/80 IPA/hexanes, Daicel Chiralpak AD column, 4.6×150 mm, 1.0 mL/min., 240 nm): 18.71 min. (R-isomer, 0.1% by area), 24.04 min. (S-isomer, 99.3% by area). HPLC (25/75 CH₃CN/0.1% aq H₃PO₄, Waters Nova-Pak C18 Column, 3.9×150 mm, 4 μm, 1.0 mL/min.): RT 4.02 min. (>99.0% by area at 210 or 240 nm). ¹H NMR (DMSO-d₆) δ: 7.44-7.69 (m, 5H), 6.86-6.94 (m, 4H), 5.75 (appt. t, 1H), 4.56 (d, 1H), 4.15 (d, 1H), 3.74 (s, 3H), 3.72 (s, 3H), 2.82-3.01 (m, 2H). ¹³C NMR (DMSO-d₆) δ 171.31, 166.86, 148.67, 148.20, 141.70, 132.30, 131.27, 127.83, 123.43, 122.79, 119.12, 111.73, 111.09, 55.49, 51.47, 46.27, 37.95. Anal. Calcd for C₁₉H₂₀N₂O₄: C, 67.05; H, 5.92; N, 8.23. Found: C, 66.85; H, 5.76; N, 8.05.

Assuming that (+)-3-(3,4-dimethoxy-phenyl)-3-(1-oxo-1,3-dihydro-isoindol-2-yl)-propionamide is (S)-3-(3,4-dimethoxy-phenyl)-3-(1-oxo-1,3-dihydro-isoindol-2-yl)-propionamide, a typical reaction scheme for preparation of (+)-3-(3,4-dimethoxy-phenyl)-3-(1-oxo-1,3-dihydro-isoindol-2-yl)-propionamide is illustrated below:

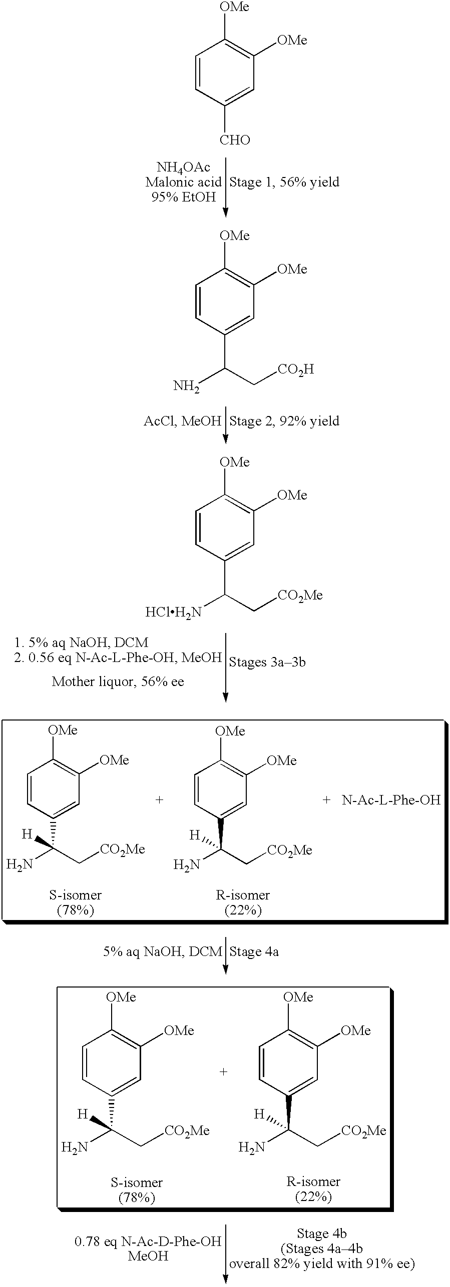

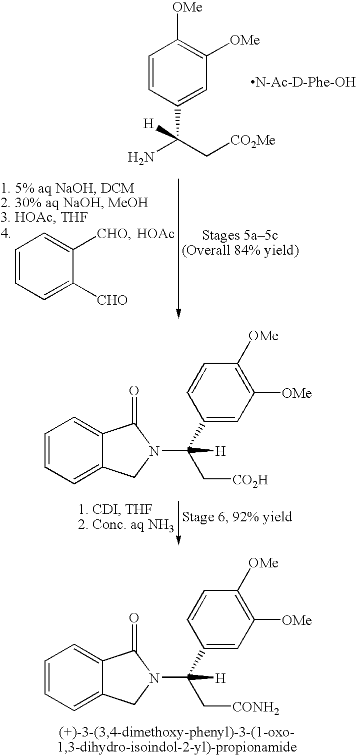

### 5.3 Example 3

### TNF-α Inhibition of (+)-3-(3,4-DIMETHOXY-PHENYL)-3-(1-OXO-1,3-DIHYDRO-ISOINDOL-2-YL)-PROPIONAMIDE

LPS-Induced TNF-α Production

Lipopolysaccharide (LPS) is an endotoxin produced by gram-negative bacteria such as E. coli which induces production of many pro-inflammatory cytokines, including TNF-α. In peripheral blood mononuclear cells (PBMC), the TNF-α produced in response to LPS is derived from monocytes, which comprise approximately 5-20% of the total PBMC. Compound is tested for the ability to inhibit LPS-induced TNF-α production from human PBMC as previously described (Muller et al. 1996, J. Med Chem. 39:3238). PBMC from normal donors are obtained by Ficoll Hypaque (Pharmacia, Piscataway, N.J., USA) density centrifugation. Cells are cultured in RPMI (Life Technologies, Grand Island, N.Y., USA) supplemented with 10% AB±human serum (Gemini Bio-products, Woodland, Calif., USA), 2 mM L-glutamine, 100 U/ml penicillin, and 100 μg/ml streptomycin (Life Technologies).

PBMC (2×10⁵ cells) are plated in 96-well flat-bottom Costar tissue culture plates (Corning, N.Y., USA) in triplicate. Cells are stimulated with LPS (Sigma, St. Louis, Mo., USA) at 100 ng/ml in the absence or presence of compounds. Compound (Celgene Corp., Warren, N.J., USA) is dissolved in DMSO (Sigma) and further dilutions are done in culture medium immediately before use. The final DMSO concentration in all samples is 0.25%. Compound is added to cells 1 hour before LPS stimulation. Cells are incubated for 18-20 hours at 37° C. in 5% CO₂ and supernatants are then collected, diluted with culture medium and assayed for TNF-α levels by ELISA (Endogen, Boston, Mass., USA).

IL-1β-Induced TNF-α Production

During the course of inflammatory diseases, TNF-α production is often stimulated by the cytokine 1L-1β, rather than by bacterially derived LPS. Compounds are tested for the ability to inhibit IL-1β-induced TNF-α production from human PBMC as described above for LPS-induced TNF-α production, except that the PBMC are isolated from source leukocyte units (Sera-Tec Biologicals, North Brunswick, N.J., USA) by centrifugation on Ficoll-Paque Plus (Amersham Pharmacia, Piscataway, N.J., USA), plated in 96-well tissue culture plates at 3×10⁵ cells/well in RPMI-1640 medium (BioWhittaker, Walkersville, Md., USA) containing 10% heat-inactivated fetal bovine serum (Hyclone), 2 mM L-glutamine, 100 U/ml penicillin, and 100 mg/ml streptomycin (complete medium), pretreated with compounds at 10, 2, 0.4, 0.08, 0.016, 0.0032, 0.00064, and 0 μM in duplicate at a final DMSO concentration of 0.1 % at 37° C. in a humidified incubator at 5% CO₂ for 1 hour, then stimulated with 50 ng/ml recombinant human IL-1β (Endogen) for 18 hours.

### 5.4 Example 4

### PDE4 Inhibition

PDE4 enzyme is purified from U937 human monocytic cells by gel filtration chromatography as previously described (Muller et al. 1998, Bioorg. & Med Chem Lett 8:2669-2674). Phosphodiesterase reactions are carried out in 50 mM Tris HCl pH 7.5, 5 mM MgCl₂, 1 μM cAMP, 10 nM [³11)-cAMP for 30 min at 30° C., terminated by boiling, treated with 1 mg/ml snake venom, and separated using AG-1XS ion exchange resin (BioRad) as described (Muller et al. 1998, Bioorg. & Med Chem Lett 8:2669-2674). Reactions consume less than 15% of available substrate.

### 5.5 Example 5

### PDE Selectivity of (+)-3-(3,4-DIMETHOXY-PHENYL)-3-(1-OXO-1,3-DIHYDRO-ISOINDOL-2-YL)-PROPIONAMIDE

The specificity of compound for specific PDE's is assessed by testing at a single concentration (100 μM) against bovine PDE1, human PDE2, PDE3, and PDE5 from human platelets (Hidaka and Asano 1976, Biochem. Biophys. Acta 429:485, and Nicholsen et al. 1991, Trends Pharmaco. Sci. 12:19).

### 5.6 Example 6

### Pharmacokinetic Data

Mean (±SD) plasma concentration-time profiles are observed for 24 hours after oral administration of (+)-3-(3,4-dimethoxy-phenyl)-3-(1-oxo-1,3-dihydro-isoindol-2-yl)-propionamide of 80 mg/kg as a single compound dosing in female rats.

### 5.7 Example 7

### 200 MG (+)-3-(3,4-DIMETHOXY-PHENYL)-3-(1-OXO-1,3-DIHYDRO-ISOINDOL-2-YL)-PROPIONAMIDE Dosage Capsule

Table I illustrates a batch formulation and single dosage formulation for a 200 mg (+)-3-(3,4-dimethoxy-phenyl)-3-(1-oxo-1,3-dihydro-isoindol-2-yl)-propionamide single dose unit, i.e., about 40 percent by weight, in for example a size #0 capsule.

TABLE I
Formulation for 200 mg capsule

| | Percent By | Quantity | Quantity |
|---|---|---|---|
| Material | Weight | (mg/tablet) | (kg/batch) |
| Compound A* | 40.0% | 200 mg | 16.80 kg |
| Pregelatinized Corn Starch, NF5 | 9.5% | 297.5 mg | 24.99 kg |
| Magnesium Stearate | 0.5% | 2.5 mg | 0.21 kg |
| Total | 100.0% | 500 mg | 42.00 kg |

*Compound A is (+)-3-(3,4-dimethoxy-phenyl)-3-(1-oxo-1,3-dihydro-isoindol-2-yl)-propionamide.

The pregelatinized corn starch (SPRESS B-820) and (+)-3-(3,4-dimethoxy-phenyl)-3-(1-oxo-1,3-dihydro-isoindol-2-yl)-propionamide components are passed through a 710 μm screen and then are loaded into a Diffusion Mixer with a baffle insert and blended for 15 minutes. The magnesium stearate is passed through a 210 μm screen and is added to the Diffusion Mixer. The blend is then encapsulated in a size #0 capsule, 500 mg per capsule (8400 capsule batch size) using a Dosator type capsule filling machine.

### 5.8 Example 8

### 100 MG Oral Dosage Form

Table II illustrates a batch formulation and a single dose unit formulation containing 100 mg of (+)-3-(3,4-dimethoxy-phenyl)-3-(1-oxo-1,3-dihydro-isoindol-2-yl)-propionamide.

TABLE II
Formulation for 100 mg tablet

| | Percent by | Quantity | Quantity |
|---|---|---|---|
| Material | Weight | (mg/tablet) | (kg/batch) |
| Compound A* | 40% | 100.00 | 20.00 |
| Microcrystalline Cellulose, NF | 53.5% | 133.75 | 26.75 |
| Pluronic F-68 Surfactant | 4.0% | 10.00 | 2.00 |
| Croscarmellose Sodium Type A, | 2.0% | 5.00 | 1.00 |
| NF | | | |
| Magnesium Stearate, NF | 0.5% | 1.25 | 0.25 |
| Total | 100.0% | 250.00 mg | 50.00 kg |

*Compound A is (+)-3-(3,4-dimethoxy-phenyl)-3-(1-oxo-1,3-dihydro-isoindol-2-yl)-propionamide.

The microcrystalline cellulose, croscarmellose sodium, and (+)-3-(3,4-dimethoxy-phenyl)-3-(1-oxo-1,3-dihydro-isoindol-2-yl)-propionamide components are passed through a #30 mesh screen (about 430 μm to about 655 μm). The Pluronic F-68® (manufactured by JRH Biosciences, Inc. of Lenexa, Kans.) surfactant is passed through a #20 mesh screen (about 457 μm to about 1041 μm). The Pluronic F-68® surfactant and 0.5 kgs of croscarmellose sodium are loaded into a 16 qt. twin shell tumble blender and are mixed for about 5 minutes. The mix is then transferred to a 3 cubic foot twin shell tumble blender where the microcrystalline cellulose is added and blended for about 5 minutes. (+)-3-(3,4-Dimethoxy-phenyl)-3-(1-oxo-1,3-dihydro-isoindol-2-yl)-propionamide is added and blended for an additional 25 minutes. This pre-blend is passed through a roller compactor with a hammer mill attached at the discharge of the roller compactor and moved back to the tumble blender. The remaining croscarmellose sodium and magnesium stearate is added to the tumble blender and blended for about 3 minutes. The final mixture is compressed on a rotary tablet press with 250 mg per tablet (200,000 tablet batch size).

### 5.9 Example 9

### Aerosol Dosage Form

A concentrate is prepared by combining (+)-3-(3,4-dimethoxy-phenyl)-3-(1-oxo-1,3-dihydro-isoindol-2-yl)-propionamide, and a 12.6 kg portion of the trichloromonofluoromethane in a sealed stainless steel vessel equipped with a high shear mixer. Mixing is carried out for about 20 minutes. The bulk suspension is then prepared in the sealed vessel by combining the concentrate with the balance of the propellants in a bulk product tank that is temperature controlled to 21° C. to 27° C. and pressure controlled to 2.8 to 4.0 BAR. 17 ml aerosol containers which have a metered valve which is designed to provide 100 inhalations of the composition of the invention. Each container is provided with the following:

| | | |
|---|---|---|
| ipratropium bromide | 0.0021 | kg |
| Compound A* | 0.0120 | kg |
| trichloromonofluoromethane | 1.6939 | g |
| dichlorodifluoromethane | 3.7028 | g |
| dichlorotetrafluoroethane | 1.5766 | g |
| total | 7.0000 | g |

*Compound A is (+)-3-(3,4-dimethoxy-phenyl)-3-(1-oxo-1,3-dihydro-isoindol-2-yl)-propionamide.

While the invention has been described with respect to the particular embodiments, it will be apparent to those skilled in the art that various changes and modifications may be made without departing from the spirit and scope of the invention as defined in the claims. Such modifications are also intended to fall within the scope of the appended claims.

What is claimed is:

1. A method of inhibiting TNF-α production which comprises contacting a cell which produces TNF-α with an effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

2. A method of inhibiting PDE4 activity which comprises contacting PDE4 with an effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

3. The method of claim 1 or 2 wherein the cell is a mammalian cell.

4. The method of claim 3 wherein the cell is a human cell.

5. A method of treating or preventing a disease or a disorder ameliorated by reduction of levels of TNF-α in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

6. A method of treating or preventing cancer which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

7. The method of claim 5 or 6 further comprising administering to a patient in need of such treatment or prevention a therapeutically or prophylactically effective amount of an alkylating agent, nitrogen mustard, a JNK inhibitor, antibiotic, antineoplastic agent, ethylenimine, methylmelamine alkyl sulfonate, nitrosourea, triazene, folic acid analog, pyrimidine analog, purine analog, vinca alkaloid, epipodophyllotoxin, steroid, a topoisomerase inhibitor, or an anti-cancer vaccine.

8. The method of claim 5, wherein the disease or disorder is diabetic retinopathy, retinopathy of prematurity, corneal graft rejection, neovascular glaucoma, retrolental fibroplasia, proliferative vitreoretinopathy, trachoma, myopia, optic pits, epidemic keratoconjunctivitis, atopic keratitis, superior limbic keratitis, pterygium keratitis sicca, sjogrens, acne rosacea, phylectenulosis, syphilis, lipid degeneration, bacterial ulcer, fungal ulcer, Herpes simplex infection, Herpes zoster infection, protozoan infection, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, sickle cell anemia, sarcoid, pseudoxanthoma elasticum, Pagets disease, vein occlusion, artery occlusion, carotid obstructive disease, chronic uveitis, chronic vitritis, Lyme's disease, Eales disease, Bechet's disease, retinitis, choroiditis, presumed ocular histoplasmosis, Bests disease, Stargarts disease, pars planitis, chronic retinal detachment, hyperviscosity syndromes, toxoplasmosis, sclerosing cholangitis, rubeosis, endotoxemia, toxic shock syndrome, osteoarthritis, retrovirus replication, wasting, meningitis, silica-induced fibrosis, asbestos-induced fibrosis, veterinary disorder, malignancy-associated hypercalcemia, stroke, circulatory shock, periodontitis, gingivitis, macrocytic anemia, refractory anemia, or 5q-syndrome.

9. The method of claims 6 wherein the cancer is a solid tumor or a blood borne tumor.

10. The method of claim 6 wherein the cancer is multiple myeloma, acute leukemia, lymphoblastic leukemia, myelogenous leukemia, lymphocytic leukemia, or myelocytic leukemia.

11. The method of claim 9 wherein the solid tumor is a tumor of the breast, colon, rectum, colorectum, kidney, or a glioma.

12. The method of claim 5 or 6 wherein the patient is a mammal.

13. The method of claim 5 or 6 wherein the enantiomerically pure (+)-3-(3,4-dimethoxy-phenyl)-3-(1-oxo-1,3-dihydro-isoindol-2-yl)-propionamide is administered parenterally, transdermally, mucosally, nasally, buccally, sublingually, topically or orally.

14. The method of claim 13 wherein the therapeutically or prophylactically effective amount is from about 1 mg to about 5,000 mg per day.

15. The method of claim 14 wherein the therapeutically or prophylactically effective amount is from about 10 mg to about 2,500 mg per day.

16. The method of claim 15 wherein the therapeutically or prophylactically effective amount is from about 100 mg to about 1,200 mg per day.

17. A method of treating or preventing a disease or disorder ameliorated by the inhibition of PDE4 in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

18. A method of controlling cAMP levels in a cell which comprises contacting a cell with an effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

19. A method of treating or preventing depression, asthma, inflammation, inflammatory skin disease, psoriasis, atopic dermatitis, contact dermatitis, rheumatoid arthritis, osteoarthritis, chronic obstructive pulmonary disease, chronic pulmonary inflammatory disease, inflammatory bowel disease, Crohn's Disease, Bechet's Disease, colitis, chronic bronchitis, allergic rhinitis, arthritis, joint inflammation, ulcerative colitis, atopic eczema, stroke, bone resorption disease, multiple sclerosis, urticaria, allergic conjunctivitis, vernal conjunctivitis, inflammation of the eye, allergic responses in the eye, eosinophilic granuloma, gouty arthritis, arthritic condition, adult respiratory distress syndrome, diabetes insipidus, keratosis, cerebral senility, multi-infarct dementia, senile dementia, memory impairment associated with Parkinson's disease, cardiac arrest, intermittent claudication, rheumatoid spondylitis, osteoarthritis, sepsis, septic shock, endotoxic shock, gram negative sepsis, toxic shock syndrome, acute respiratory distress syndrome, cerebral malaria, silicosis, pulmonary sarcoidosis, reperfusion injury, graft vs host reaction, allograft rejection, infection-related fever, myalgia, malaria, HIV, AIDS, ARC, cachexia, keloid formation, scar tissue formation, pyresis, systemic lupus erythematosus, type 1 diabetes mellitus, anaphylactoid purpura nephritis, chronic glomerulonephritis, leukemia, tarditive dyskinesia, yeast infection, fungal infection, condition requiring gastroprotection, or neurogenic inflammatory disease associated with irritation or pain, which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

20. A method of treating or preventing myelodysplastic syndrome in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

21. A method of treating or preventing myeloproliferative disease in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

22. A method of treating or preventing pain in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

23. A method of treating or preventing-macular degeneration in a patient which comprises administering to a patient in need of such treatment or prevention a therapeutically or prophylactically effective amount of enantiomerically pure (+)-3-(3,4-dimethoxy-phenyl)-3-(1-oxo-1,3-dihydro-isoindol-2-yl)-propionamide, or a pharmaceutically acceptable salt or solvate thereof.

24. The method of any one of claims 17 to 23 further comprising administering to a patient in need of such treatment or prevention a therapeutically or prophylactically effective amount of an antihistamine, anti-inflammatory drug, non-steroid anti-inflammatory drug, steroid, anti-cancer agent, hematopoietic growth factor, cytokine, stem call transplantation, or kinase inhibitor.

25. The method of claim 17 wherein the disease or disorder is respiratory disease, asthma, allergic rhinitis, inflammation or chronic pulmonary inflammatory disease.

26. The method of claim 17 wherein the disease or disorder is chronic obstructive pulmonary disease.

27. The method of any one of claims 17 to 23 wherein the patient is a mammal.

28. The method of any one of claims 17 to 23 wherein the enantiomerically pure (+)-3-(3,4-dimethoxy-phenyl)-3-(1-oxo-1,3-dihydro-isoindol-2-yl)-propionamide is administered parenterally, transdermally, mucosally, nasally, buccally, sublingually, topically, or orally.

29. The method of any one of claims 17 to 23 wherein the therapeutically or prophylactically effective amount is from about 1 mg to about 5,000 mg per day.

30. The method of claim 29 wherein the therapeutically or prophylactically effective amount is from about 10 mg to about 2,500 mg per day.

31. The method of claim 30 wherein the therapeutically or prophylactically effective amount is from about 100 mg to about 1,200 mg per day.

32. The method of claim 30 or 31, wherein the enantiomerically pure (+)-3-(3,4-dimethoxy-phenyl)-3-(1-oxo-1,3-dihydro-isoindol-2-yl)-propionamide is administered twice a day.

33. A pharmaceutical composition comprising enantiomerically pure (+)-3-(3,4-dimethoxy-phenyl)-3-(1-oxo-1,3-dihydro-isoindol-2-yl)-propionamide, or a pharmaceutically acceptable salt or solvate thereof; and a pharmaceutically acceptable carrier, excipient or diluent.

34. The pharmaceutical composition of claim 33 wherein said pharmaceutical composition is suitable for parenteral, transdermal, mucosal, nasal, buccal, sublingual, topical or oral administration to a patient.

35. Enantiomerically pure (+)-3-(3,4-dimethoxy-phenyl)-3-(1-oxo-1,3-dihydro-isoindol-2-yl)-propionamide, substantially free of its (−) isomer, or a pharmaceutically acceptable salt or solvate thereof.

36. The enantiomerically pure salt of claim 35 which is a chiral amino acid salt.

37. The enantiomerically pure salt of claim 36 wherein the chiral amino acid is the D isomer of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, N-acetyl-phenylalanine or N-acetyl-leucine.

38. A method of producing enantiomerically pure (+)-3-(3,4-dimethoxy-phenyl)-3-(1-oxo-1,3-dihydro-isoindol-2-yl)-propionamide which comprises:
(a) contacting (S)-3-amino-3-(3,4-dimethoxyphenyl)propionic acid with phthalic dicarboxaldehyde under a condition sufficient to form (S)-3-(3,4-dimethoxyphenyl)-3-(1-oxo-1,3-dihydro-isoindol-2-yl)propionic acid; and
(b) reducing the (S)-3-(3,4-dimethoxyphenyl)-3-(1-oxo-1,3-dihydro-isoindol-2-yl)propionic acid under a condition sufficient to form (+)-3-(3,4-dimethoxy-phenyl)-3-(1-oxo-1,3-dihydro-isoindol-2-yl)-propionamide.

39. The method of claim 38, wherein a chiral amino acid salt of (S)-methyl 3-amino-3-(3,4-dimethoxyphenyl)-propionate is contacted with methylene chloride and tetrahydrofuran under conditions sufficient to form (S)-3-amino-3-(3,4-dimethoxyphenyl)propionic acid.

40. The method of claim 39, wherein methyl 3-amino-3-(3,4-dimethoxyphenyl)-propionate is contacted with a chiral amino acid under a condition sufficient to form the chiral amino acid salt of (S)-methyl 3-amino-3-(3,4-dimethoxyphenyl)propionate.

41. The method of claim 39 or 40 wherein the chiral amino acid is the D isomer of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, N-acetyl-phenylalanine or N-acetyl-leucine.

42. The method of claim 41 wherein the chiral amino acid salt is N-acetyl-D-phenylalanine.

43. An enantiomerically pure salt of (+)-methyl 3-amino-3-(3,4-dimethoxyphenyl)propionate.

44. The enantiomerically pure salt of claim 43 which is a chiral amino acid salt.

45. The enantiomerically pure salt of claim 44 wherein the chiral amino acid is the D isomer of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, N-acetyl-phenylalanine or N-acetyl-leucine.

46. (+)-Methyl 3-amino-3-(3,4-dimethoxyphenyl)propionate N-acetyl-D-phenylalanine salt.
